(19) United States
(12) Patent Application Publication
(10) Pub. No.: US 2004/0238095 A1
(43) Pub. Date: Dec. 2, 2004

(54) Method for attaching mechanical fasteners to absorbent articles and the resulting absorbent articles

(75) Inventor: Betsy Johnson, Atlanta, GA (US)

Correspondence Address: HUNTON & WILLIAMS, ATTN: CHRISTOPHER C. CAMPBELL, SUITE 1200, 1900 K STREET, N.W., WASHINGTON, DC, 20006, US

(73) Assignee: Paragon Trade Brands, Inc.

(21) Appl. No.: 10/865,962

(22) Filed: Jun. 14, 2004

Related U.S. Application Data
(63) Continuation of application No. 09/685,610, filed on Oct. 11, 2000.
(60) Provisional application No. 60/159,562, filed on Oct. 15, 1999.

(51) Int. Cl. A44B 19/00; A41H 37/00; A61F 13/15

(52) U.S. Cl. 156/66; 604/392

(57) ABSTRACT

The present invention relates to methods for attaching mechanical fasteners to absorbent articles using slow-crystallizing hot melt adhesives under conditions sufficient to result in a mechanical fastener/absorbent article bond static shear strength of at least about 50 min/kg. Preferred mechanical fasteners include hook-type fastener tabs. The present invention provides an efficient, reliable method for attaching mechanical fasteners to absorbent articles which is superior to previous techniques. The present invention also relates to absorbent articles produced by the described method.

### RELATED APPLICATIONS

This application claims priority to U.S. Provisional Patent Application Serial No. 60/159,562, filed Oct. 15, 1999, which is incorporated herein by reference.

### FIELD OF THE INVENTION

This invention relates to methods for attaching mechanical fasteners to absorbent articles and the resulting absorbent articles. In particular, the present invention relates to methods for attaching hook-type fastener tabs to absorbent articles using slow-crystallizing hot melt adhesives.

### BACKGROUND OF THE INVENTION

The use of mechanical fastening means on absorbent articles, such as diapers, training pants, adult incontinent products, feminine care products, and the like, are well known. A common type of mechanical fastener employed on absorbent articles is a hook-and-loop type fastener in which a hook or hook-type fastener tab is provided which is adapted for releasably engaging with a loop or loop-like material. Such hook-and-loop type fasteners are also well known in the prior art. Other well known types of mechanical fasteners include snaps, buttons, zippers, mushroom fasteners and the like.

While the use of mechanical fasteners, such as hook-and-loop type fasteners, on absorbent articles is known, they have limitations. This is due, in part, to the difficulty in reliably attaching the hook-type fastener tabs to absorbent articles using conventional techniques. Thus, in order to render the use of mechanical fasteners, such as hook-and-loop type fasteners, on disposable absorbent articles more feasible, it is desirable to provide an improved method for attaching the hook-type fastener tabs to absorbent articles.

### SUMMARY OF THE INVENTION

The present invention overcomes the aforementioned difficulty by supplying an effective and reliable method for attaching mechanical fasteners to absorbent articles using slow-crystallizing hot melt adhesives under conditions sufficient to result in a mechanical fastener/absorbent article bond static shear strength of at least about 50 min/kg. More particularly, the present invention relates to a method for attaching a hook-type fastener tab to an absorbent article using a slow-crystallizing hot melt adhesive and the resulting absorbent article.

In a preferred embodiment, the present invention relates to a method for attaching a mechanical fastener to an absorbent article comprising the steps of:

a) providing said absorbent article;

b) providing said mechanical fastener;

c) applying a slow-crystallizing hot melt adhesive to said absorbent article in a target area; and

d) attaching said mechanical fastener to said absorbent article in the target area with slow-crystallizing hot melt adhesive under conditions sufficient to result in a mechanical fastener/absorbent article bond static shear strength of at least about 50 min/kg.

In another embodiment, the present invention relates to an absorbent article produced according to the above described method comprising:

a) a liquid previous topsheet;

b) a liquid impervious backsheet joined to said topsheet;

c) an absorbent core positioned between said topsheet and said backsheet; and

d) at least one mechanical fastener positioned so as to secure the absorbent article to an intended user, wherein the mechanical fastener is attached to said absorbent article using a slow-crystallizing hot melt adhesive under conditions sufficient to result in a mechanical fastener/absorbent article bond static shear strength of at least about 50 min/kg.

### BRIEF DESCRIPTION OF THE DRAWINGS

The present invention will be more fully understood and further advantages will become apparent when reference is made to the following detailed description of the invention and the accompanying drawings. The drawings are merely representative and are not intended to limit the scope of the appended claims.

FIG. 1 is a partially cut away plan view of an absorbent article having mechanical fasteners according to the present invention;

FIG. 2 is a perspective view of an absorbent article according to the present invention which illustrates an alternative attachment of the mechanical fasteners of the present invention;

FIG. 3 illustrates a test sample prepared for the static shear strength test described below in connection with the example.

### DETAILED DESCRIPTION OF THE PREFERRED EMBODIMENTS

The present invention relates to a process for attaching mechanical fasteners to absorbent articles and absorbent articles produced by the same. The term “absorbent article” is intended to refer to any article intended to absorb discharged body fluids. Examples of absorbent articles include diapers, adult incontinence products, training pants, feminine napkins, wound dressings, and the like. For ease of understanding, much of the following description of the present invention will be made in terms of the process of attaching hook-type fastener tabs to diapers and the diapers made by such processes. Nonetheless, it is to be understood that the present invention is equally suited to any mechanical fastener and any absorbent article.

In one embodiment, the present invention refers to a method for attaching mechanical fasteners to absorbent articles using slow-crystallizing hot melt adhesives. The method comprises the steps of providing an absorbent article; providing a mechanical fastener; applying a slow-crystallizing hot melt adhesive to said absorbent article in a target area; and attaching the mechanical fastener to the absorbent article in the target area with the slow-crystallizing hot melt adhesive.

Methods for applying the slow-crystallizing hot melt adhesive to the target area of the absorbent article are similar to traditional hot melt adhesive application techniques. The adhesive is applied in a molten state directly to the target area of the absorbent article, and while the adhesive is still in a molten state, the mechanical fastener is applied under pressure to the exposed adhesive surface of the target area. When the adhesive cools to ambient temperature, it crystallizes and its viscosity increases so as to form an interfacial adhesive bond between the mechanical fastener and the absorbent article.

While not being bound by theory, it is believed that the crystalline form of the adhesive increases the cohesive and adhesive properties of the adhesive, which thereby results in greater bond strength. As opposed to typical hot melt adhesive bonds which tend to soften over time under heat and stress, the crystalline structure of the adhesive bonds formed according to the method of the present invention are more resistant to the effects of heat and stress. As such, it was unexpectedly discovered that when mechanical fasteners are attached to absorbent articles according to the method of the present invention, a mechanical fastener/absorbent article bond static shear strength of at least about 50 min/kg, more preferably at least about 70 min/kg, and most preferably of at least about 200 min/kg, is attained. As used herein, static shear strength is determined at 120° F. according to the method described in the detailed examples.

Slow-crystallizing hot melt adhesives useful in the present invention to attain such static shear bond strengths are available from H. B. Fuller under the product name HL-1696. Further, suitable slow-crystallizing hot melt adhesives are described in U.S. Pat. Nos. 5,624,986; 5,627,229; 5,853,864; and 5,863,977 which are herein incorporated by reference in a manner consistent with this disclosure.

The slow-crystallizing hot melt adhesive can be applied to the target area of the absorbent article using a process selected from the group consisting of slot coating, solid shim coating, comb shim coating, and spray-on techniques. Further, the slow-crystallizing hot melt adhesive can be applied to the target area of the absorbent article at any temperature suitable to melt the adhesive. Preferably, the temperature is sufficient to melt the adhesive, but below about 325° F.

The slow-crystallizing hot melt adhesive is applied to the target area of the absorbent article in an amount sufficient to secure the mechanical fastener to the absorbent article with a static shear strength bond of at least about 50 min/kg. Preferably, the slow-crystallizing hot melt adhesive is applied to the target area of the absorbent article in an amount of less than about 0.045 grams/target area.

Further, the use of additional bonding techniques, such as ultrasonic bonding and various forms of mechanical bonding known in the art, in conjunction with slow-crystallizing hot melt adhesives are well within the level of ordinary skill in the art and are therefore considered within the scope of the present invention.

Those skilled in the art will recognize that the diaper 20 generally comprises a backsheet 22, a topsheet 24, and an absorbent core 26 located between the backsheet 22 and the topsheet 24. Leg elastics 28 are located generally at the longitudinal edges of the diaper 20. The disposable diaper 20 may further comprise a front waist elastic 30 and a rear waist elastic 32. These component parts can be joined together by any method known in the art. As representatively illustrated, the disposable diaper 20 also includes mechanical fasteners 34 and 36, which are attached to the diaper according to the method of the present invention.

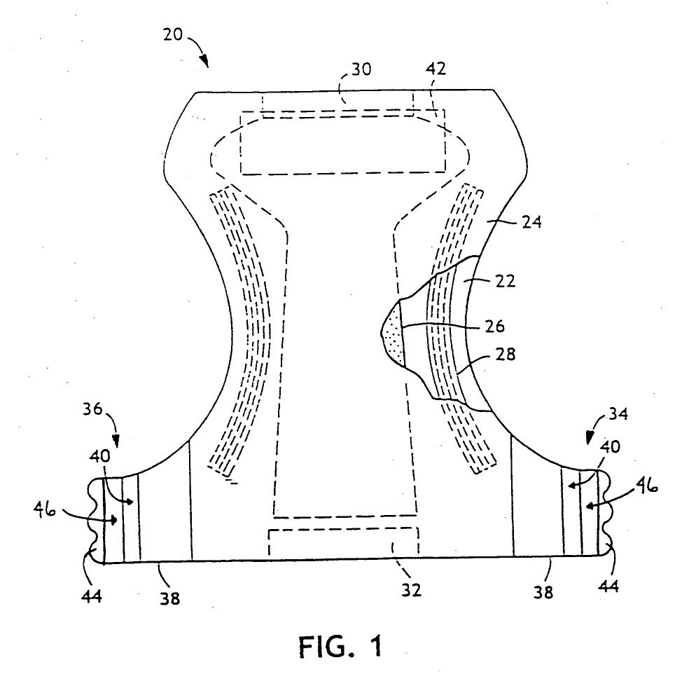

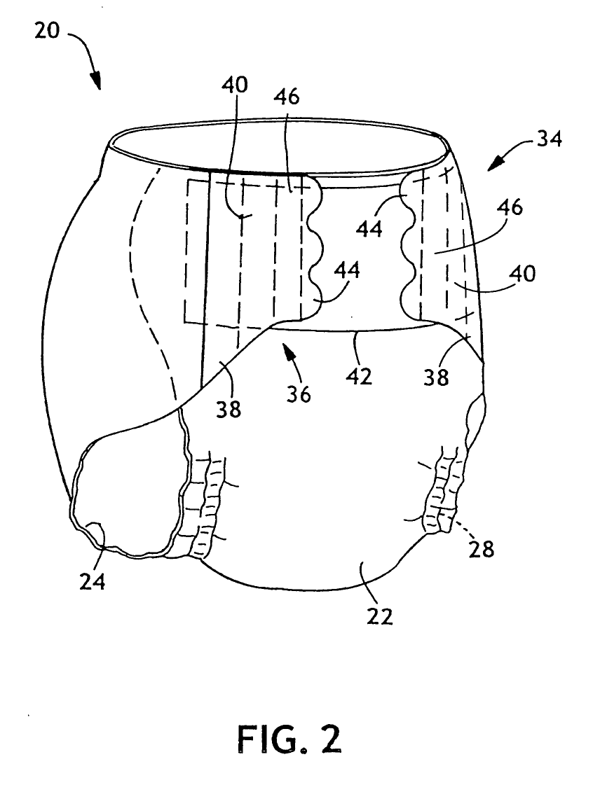

The backsheet 22 of the disposable diaper 20, as representatively illustrated in FIGS. 1-2, may be formed from any suitable material known in the art. The backsheet 22 can be either liquid permeable or liquid impermeable. However, it is generally preferred that the backsheet 22 be formed from a material which is substantially impermeable to liquids. A typical backsheet can be manufactured from a thin plastic film or other flexible liquid-impermeable material. If it is desired to present the backsheet 22 with a more cloth-like feeling, the backsheet 22 may comprise a polyethylene film having a nonwoven web laminated to the outer surface thereof, such as a spunbond web of polyolefin fibers. Methods of forming such cloth-like backsheets are known to those skilled in the art.

Further, the backsheet 22 may be formed of a woven or nonwoven fibrous web layer which has been totally or partially constructed or treated to impart a desired level of liquid impermeability to selected regions that are adjacent or proximate the absorbent core 26. Still further, the backsheet 22 may optionally be composed of a micro-porous “breathable” material which permits vapors to escape from the absorbent core 26 while still preventing liquid exudates from passing through the backsheet 22.

The topsheet 24 of the disposable diaper 20, as representatively illustrated in FIGS. 1-2, suitably presents a body-facing surface which is compliant, soft feeling and non-irritating to the wearer's skin. Further, the topsheet 24 is preferably less hydrophilic than the absorbent core 26, to present a relatively dry surface to the wearer, and may be sufficiently porous to be liquid permeable, permitting liquid to readily penetrate through its thickness. If the topsheet is formed from a substantially hydrophobic material, and the hydrophobic material may, optionally, be treated with a surfactant or otherwise processed to impart a desired level of wettability and hydrophilicity.

The topsheet 24 is preferably formed from polymeric fabrics such as polyolefin nonwoven fabrics. Common polyolefin nonwoven fabrics include polypropylene and polyethylene spunbonded fabrics. Such fabrics are typically produced by processes disclosed in the following patents: U.S. Pat. No. 4,340,563 to Appel et al., U.S. Pat. No. 3,692,618 to Dorschner et al., U.S. Pat. No. 3,338,992 to Kinney, U.S. Pat. No. 3,341,394 to Kinney, U.S. Pat. No. 3,502,538 to Levy, U.S. Pat. No. 3,502,763 to Hartmann, U.S. Pat. No. 3,909,009 to Hartmann, and U.S. Pat. No. 3,542,615 to Dobo et al., all of which are hereby incorporated by reference. Additionally, the topsheet 24 of the present invention can be formed from nonwoven bicomponent polymeric fabrics. Nonwoven bicomponent fabrics are typically produced by processes such as are disclosed in U.S. Pat. No. 3,423,266 to Davies et al. and U.S. Pat. No. 3,595,731 to Davies, et al., which are hereby incorporated by reference.

The absorbent core 26 can be formed from any absorbent materials known in the art, such as fluff pulp, superabsorbent polymers, or laminates of absorbent materials. For instance, the absorbent core 26 can preferably comprise a thin, high density, folded absorbent laminate that contains superabsorbent polymer (SAP) and stabilization additives. Preferably, the SAP and the stabilization additives are included in an absorption layer, which is sandwiched between an upper layer of highly porous, liquid permeable material (the “upper layer”) and a lower layer of a high wet strength material (the “lower layer”) that can be made substantially liquid impervious by application of a nearly coherent film of adhesive, i.e., at a high adhesive coverage.

Except as illustrated and described herein, the diaper 20 may be substantially similar to the diaper disclosed in Huffman et al. U.S. Pat. No. 5,403,301, or in Chmielewski U.S. Pat. No. 5,891,120, both assigned to the assignee of the present invention, the disclosures of which are incorporated herein by reference in a manner consistent with this disclosure.

As representatively illustrated in FIGS. 1-2, the disposable diaper 20 includes mechanical fasteners 34 and 36. The mechanical fasteners 34 and 36 include the hook-type fastener tab of a hook-and-loop type mechanical fastener, as are well known to those skilled in the art. Other fasteners which may be used in the present invention include cohesive materials, snaps, buttons and the like. As used herein, reference to a mechanical fastener refers to at least one component of a combination of a first fastening element which is adapted to mechanically interlock with a second fastening element. For example, the first and second fastening elements of the mechanical fasteners may include the hook-type fastening tab and loop material of a hook-and-loop type fastener or the male and female snap portions of a snap type fastener.

For ease of understanding, much of the following description of the present invention will be made in terms of the process of attaching hook-type fastener tabs on disposable diapers. Nonetheless, it is to be understood that the process described is equally suited for use in attaching any other type of mechanical fasteners.

Hook-and-loop type fasteners are known to those skilled in the art and are generally available from YKK Corporation of America, Velcro Industries, and Minnesota Mining and Mineral Co. The mechanical fasteners 34 and 36, as representatively illustrated in FIGS. 1-2, generally comprise a hook-type fastener tab 44 and/or a loop material 42. The mechanical fasteners 34 and 36 may also preferably include a film substrate 46 bridging the hook-type fastener tab 44 to the diaper 20. The loop or loop-type material 42 generally comprises a woven or nonwoven material defining individual loops of material which can interlock with the hook or hook-like material 44.

In general, the first fastening element or hook-type fastening tab 44 will be attached to the rear periphery of the disposable diaper 20, and the second fastening element or loop material 42 will be attached to the outer surface of the disposable diaper 20, near a front edge of the diaper. Alternatively, the hook-type fastening tab 44 may be attached to the front periphery of the disposable diaper 20, and the loop material may be attached to the outer surface of the disposable diaper 20, near a rear edge of the diaper. Those skilled in the art will appreciate that the loop material 42, with which the hook-type fastening tab 44 is intended to engage, will be positioned on the disposable diaper 20 such that the mechanical fasteners 34 and 36 can be utilized to attach the disposable diaper 20 about the waist of a wearer.

As representatively illustrated in FIGS. 1-2, the disposable diaper 20 of the present invention may also include at least one ear tab 38. Typically, the ear tab 38 is attached to the rear periphery of the diaper 20 by any method known in the art. In such a configuration, the hook-type fastener tab 44, can be attached directly to the ear tab 38 using the method of the present invention. The ear tab 38 can be made of any material which provides the desired fastening of the disposable diaper 20 about the waist of the wearer. The ear tab 38 may be formed out of a material such as that used to form the backsheet and/or topsheet. Suitable materials from which the ear tab may be formed include films, nonwoven materials, woven or knit materials, foams, and composites and laminates of the above materials.

Instead of attaching directly to the hook-type fastener tab 44, the ear tab 38 may be attached to an elastomer substrate 40, which is in turn directly attached to the hook-type fastener tab 44. In such a configuration, the diaper 20, the ear tab 38, and the elastomer substrate 40 can all be attached using any method known in the art (including the method of the present invention), while the hook-type fastener tab 44 is attached to the elastomer substrate 40 using the method according to the present invention.

Alternatively, as shown in FIGS. 1-2, the ear tab 38 can be attached to an elastomer substrate 40, the elastomer substrate 40 can then be attached to a film substrate 46, and the film substrate 46 can in turn be attached to a hook-type fastener tab 44. Again, in such an embodiment, the diaper 20, the ear tab 38, the elastomer substrate 40, and the film substrate can all be attached using any method known in the art (including the method of the present invention), while the hook-type fastener tab 44 is attached to the film substrate 46 using the method of the present invention.

The elastomer substrate 40 can be formed from any known elastic material, such as elastomer films, e.g., natural or synthetic rubber; elastomer strands, e.g., Lycra.TM. strands; elastomer foams, e.g., urethane foams; elastomer nonwoven materials; laminates or composites of such elastomer materials with other elastomer or non-elastomer materials; and the like. Likewise, the film substrate 46 can be formed from any suitable material known in the art.

As will be appreciated, several permutations and combinations of the methods of manufacture and the resulting absorbent articles of the present invention are possible. Without intending to limit the claimed invention or equivalents thereof, some of the preferred exemplary methods and absorbent articles include those described in the following examples.

### EXAMPLES

Fastener Tab Static Shear Strength

This test measures the static shear strength of a mechanical fastener/absorbent article bond. The test utilizes a static load at an elevated temperature to simulate extended wear conditions. The test is considered an accelerated test, because the test load and temperatures are intentionally high to reduce the test time. The particular mechanical fastener tested in the present example included a hook-type fastener tab attached to a film substrate, which is in turn attached to an elastomer substrate which is in turn attached to backsheet material. The control samples utilized a tradition hot melt adhesive to secure the component pieces together. The test samples utilized a slow-crystallizing hot melt adhesive according to the present invention to secure the component pieces together.

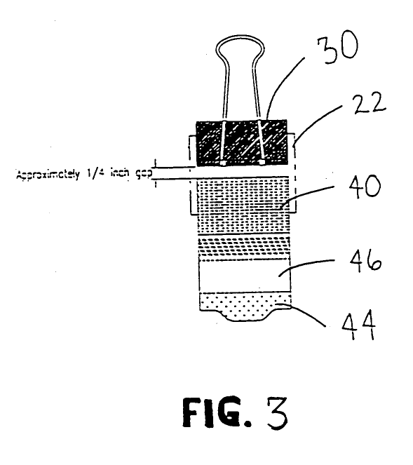

The test is performed by first placing the mechanical fastener to be tested into a clamp as shown in FIG. 3. FIG. 3 shows a clamp 30 attaching to the backsheet 22 about ¼ inch from the start of the elastomer substrate. As shown in FIG. 3, the elastomer substrate 40 is attached to the film substrate 46, which is in turn attached to the hook-type fastener tab 44. Loop material with a predetermined amount of weight approximately uniformly distributed is then attached to the hook-type fastener tab. The entire sample is then placed in an incubator and monitored until the adhesive bond securing the hook-type fastener tab to the film substrate is compromised and the hook-type fastener tab, loop material, and weights separate from the film substrate. The particular conditions utilized in the present example are as indicated in Table 1.

TABLE 1

| | | | | | | | Static Shear |
|---|---|---|---|---|---|---|---|
| | Adhesive | Hook to Film Bond | | Elastomer to Film Bond | | | Strength |
| Sample | Type | Add-On* | Temp** | Type | Add-On* | Temp** | min/1.2 kg (min/kg) |
| Control 1 | ATO Findley | 100% | 325° F. | laminate | 100% | 325° F. | 50 (41.7) |
| | H2587-01 | | | | | | |
| Control 2 | ATO Findley | 100% | 325° F. | nonwoven | 100% | 325° F. | 35 (29.2) |
| | H2587-01 | | | | | | |
| Sample 1 | HB Fuller | 50% | 325° F. | laminate | 50% | 325° F. | 220 (183.3) |
| | HL-1696 | | | | | | |
| Sample 2 | HB Fuller | 75% | 325° F. | laminate | 75% | 325° F. | 270 (225) |
| | HL-1696 | | | | | | |
| Sample 3 | HB Fuller | 100% | 325° F. | laminate | 100% | 325° F. | 720 (600) |
| | HL-1696 | | | | | | |
| Sample 4 | HB Fuller | 50% | 325° F. | nonwoven | 50% | 325° F. | 70 (58.3) |
| | HL-1696 | | | | | | |
| Sample 5 | HB Fuller | 75% | 325° F. | nonwoven | 75% | 325° F. | 220 (183.3) |
| | HL-1696 | | | | | | |
| Sample 6 | HB Fuller | 100% | 325° F. | nonwoven | 100% | 325° F. | 310 (258.3) |
| | HL-1696 | | | | | | |
| Sample 7 | HB Fuller | 50% | 325° F. | nonwoven | 50% | 325° F. | 210 (175) |
| | HL-1696 | | | | | | |
| Sample 8 | HB Fuller | 75% | 325° F. | nonwoven | 75% | 325° F. | 400 (333.3) |
| | HL-1696 | | | | | | |
| Sample 9 | HB Fuller | 100% | 325° F. | nonwoven | 100% | 325° F. | 720 (600) |
| | HL-1696 | | | | | | |
| Sample 10 | HB Fuller | 50% | 325° F. | laminate | 50% | 325° F. | 440 (366.7) |
| | HL-1696 | | | | | | |
| Sample 11 | HB Fuller | 75% | 325° F. | laminate | 75% | 325° F. | 720 (600) |
| | HL-1696 | | | | | | |
| Sample 12 | HB Fuller | 100% | 325° F. | laminate | 100% | 325° F. | 720 (600) |
| | HL-1696 | | | | | | |
| Sample 13 | HB Fuller | 50% | 300° F. | laminate | 50% | 300° F. | 340 (283.3) |
| | HL-1696 | | | | | | |
| Sample 14 | HB Fuller | 75% | 300° F. | laminate | 75% | 300° F. | 720 (600) |
| | HL-1696 | | | | | | |
| Sample 15 | HB Fuller | 100% | 300° F. | laminate | 100% | 300° F. | 720 (600) |
| | HL-1696 | | | | | | |
| Sample 16 | HB Fuller | 50% | 300° F. | nonwoven | 50% | 300° F. | 50 (41.7) |
| | HL-1696 | | | | | | |
| Sample 17 | HB Fuller | 75% | 300° F. | nonwoven | 75% | 300° F. | 340 (283.3) |
| | HL-1696 | | | | | | |
| Sample 18 | HB Fuller | 100% | 300° F. | nonwoven | 100% | 300° F. | 620 (516.7) |
| | HL-1696 | | | | | | |
| Sample 19 | HB Fuller | 100% | 275° F. | nonwoven | 100% | 275° F. | 420 (350) |
| | HL-1696 | | | | | | |

Note:
*Add-On refers to a relative amount of adhesive added to the target bonding area compared to an amount typically used in the prior art (e.g., 50% Add-On uses ½ the amount of adhesive used in the control sample).
**Temp refers to the temperature at which the adhesive is applied to the film substrate or elastomer substrate for bonding to the hook-type fastener tab and film substrate, respectively.
***The Static Shear Strength was determined at a temperature of 120° F.

As Table 1 demonstrates, even at lower processing temperatures and adhesive amounts, the method of the present invention unexpectedly out performs traditional assembly techniques.

Although the invention has been described in connection with the preferred embodiments, these embodiments are not intended to limit the invention. Those skilled in the art will readily appreciate that various modifications may be made to the preferred embodiments without departing from the scope and spirit of the invention as defined by the appended claims.

1-10. (cancelled)

11. A method for attaching a mechanical fastener to an absorbent article comprising the steps of:
a) providing said absorbent article;
b) providing said mechanical fastener;
c) applying a slow-crystallizing hot melt adhesive to said absorbent article in a target area; and
d) attaching said mechanical fastener to said absorbent article in the target area with said slow-crystallizing hot melt adhesive under conditions sufficient to result in a mechanical fastener/absorbent article bond static shear strength of at least about 60 min/1.2 kg.

12. The method according to claim 1 wherein said mechanical fastener/absorbent article bond static shear strength is at least about 240 min/1.2 kg.

13. The method according to claim 1 wherein said absorbent article comprises an ear tab, an elastomer substrate attached to said ear tab, and a film substrate attached to said elastomer substrate, wherein said target area with slow-crystallizing hot melt adhesive is located on said film substrate.

14. The method according to claim 3 wherein said elastomer substrate is selected from the group consisting of a nonwoven and a laminate structure.

15. The method according to claim 1 wherein said slow-crystallizing hot melt adhesive is applied to the target area of said absorbent article at a temperature below about 325° F.

16. The method according to claim 1 wherein said slow-crystallizing hot melt adhesive is applied to the target area of said absorbent article using a process selected from the group consisting of slot coating, solid shim coating, comb shim coating, and spray-on techniques.

17. The method according to claim 1 wherein said slow-crystallizing hot melt adhesive is applied to the target area of said absorbent article in an amount less than about 0.045 grams/target area.

18. An absorbent article comprising:
a) a liquid pervious topsheet;
b) a liquid impervious backsheet joined to said topsheet;
c) an absorbent core positioned between said topsheet and said backsheet; and
d) at least one mechanical fastener positioned so as to secure the absorbent article to an intended user, wherein the mechanical fastener is attached to said absorbent article using a slow-crystallizing hot melt adhesive under conditions sufficient to result in a mechanical fastener/absorbent article bond static shear strength of at least about 60 min/1.2 kg.

19. The absorbent article of claim 8 further comprising:
a) an ear tab attached to said backsheet;
b) an elastomer substrate attached to said ear tab; and
c) a film substrate attached to said elastomer substrate, wherein said mechanical fastener is attached to said film substrate.

20. The absorbent article of claim 8 wherein said mechanical fastener is a hook fastener tab.
